(19) United States
(12) Patent Application Publication
(10) Pub. No.: US 2013/0023661 A1
(43) Pub. Date: Jan. 24, 2013

(54) FLUORINATING REAGENTS WITH (PERFLUORALKYL) FLUOROPHOSPHATE ANION

(71) Applicants: Nikolai (Mykola) Ignatyev, Duisburg (DE); Aleksandra Atatrah, Augsburg (DE); Peter Barthen, Rheinberg (DE); Walter Frank, Wuppertal (DE)

(72) Inventors: Nikolai (Mykola) Ignatyev, Duisburg (DE); Aleksandra Atatrah, Augsburg (DE); Peter Barthen, Rheinberg (DE); Walter Frank, Wuppertal (DE)

(73) Assignee: MERCK PATENT GESELLSCHAFT MIT BESCHRANKTER HAFTUNG, Darmstadt (DE)

(21) Appl. No.: 13/639,627

(22) PCT Filed: Mar. 7, 2011
(86) PCT No.: PCT/EP2011/001101

§ 371 Date: Oct. 5, 2012

(30) Foreign Application Priority Data
Apr. 6, 2010 (DE) 10 2010 013 971.8

(51) Int. Cl. C07D 487/08; C07C 45/63; C07D 213/61

(52) U.S. Cl. 544/351; 546/345; 568/323

(57) ABSTRACT

The present invention relates to the use of compounds of the formula (I) as fluorinating reagents, to a process for the fluorination of organic compounds by reaction thereof with compounds of the formula (I), and to compounds of the formula (I).

Electrophilic fluorination is of major importance for the synthesis of novel fluorinated organic molecules [Organofluorine Chemistry. Principles and Commercial Application, R. E. Banks, B. E. Smart, J. C. Tatlow (Eds.), Plenum Press, N.Y., London, 1994, p. 42]. Molecular fluorine (F₂) has been employed for many years for the direct fluorination of organic compounds. However, this reaction is difficult to control owing to the high reactivity of F₂. In general, a mixture of different fluorinated by-products with a relatively low content of the desired product forms in the highly exothermic reaction. A further disadvantage on use of gaseous fluorine are its high toxicity, the low boiling point and its poor solubility in organic solvents. In order to counter these disadvantages, a number of fluorinated reagents has been developed, for example fluoroxytrifluoromethane (CF₃OF), acetyl hypofluorite (CH₃C(O)OF), caesium fluoroxysulfate (CsSO₄F), xenon difluoride (XeF₂), perchloryl fluoride (FClO₃) [P. Kirsch, Modern Fluoroorganic Chemistry. Synthesis, Reactivity, Applications, WILEY-VCH, 2004]. Disadvantages of these reagents are their low stability and the hazardous handling. For these reasons, novel stable fluorinated reagents which can be employed for electrophilic fluorination are constantly being developed.

The most important advance was in the development of so-called “NF” reagents of the formula R₂N-F and R₃N⁺-F.

EP 0478210 A1 discloses N-fluorinated 1,4-diazabicyclo[2.2.2]octane derivatives having anions such as halides, fluorosulfates, alkanesulfonates, alkylsulfates, perfluoroalkanesulfonates, in particulartriflates (CF₃SO₃⁻) and nonaflates (C₄F₉SO₃⁻), arenesulfonates, in particular tosylates, alkane-carboxylates, perfluoroalkanecarboxylates, tetrafluoroborates (BF₄⁻), tetraphenylborates, hexafluorophosphates (PF₆⁻), hexafluoroantimonates, chlorates and sulfates.

Some NF reagents of this type are already commercially available today, for example 1-chloromethyl-4-fluorodiazoniabicyclo[2.2.2]octane bistetrafluoroborate (Selectfluor™, F-TEDA-BF₄; compound 1) [R. E. Banks, 1998, J. Fluorine Chem. 87: 1-17; J. M. Hart, R. J. Syvret, 1999, J. Fluorine Chem. 100: 157-161]

or N-fluoropyridinium salts (NFPy; compound 2) [T. Umemoto, G. Tomiizawa, H. Hachisuka, M. Gitano, 1996, J. Fluorine Chem. 77: 161-168]:

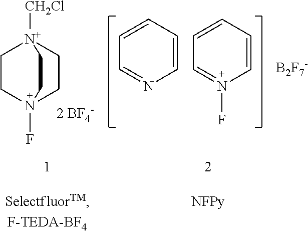

These reagents have already been employed in various reactions and exhibit good activity in the electrophilic fluorination of organic substances, for example of aromatic compounds, thioethers, enol esters, thioglucosides, alkenes, heterocyclic compounds or compounds containing an activated methylene group [G. S. Lai, G. P. Pez, R. G. Syvret, 1996, Chem. Rev. 96: 1737-1755; P. T. Nyffeler, S. G. Duron, M. D. Burkart, St. P. Vincent, Ch. -H. Wong, 2005, Angew. Chem. 117: 196-217].

Fluorination using F-TEDA-BF₄ is carried out under mild conditions, with in general monofluorinated products forming in good yield. It has been shown in studies that the fluorination force of the NF reagents is dependent principally on the structure of the R₃N⁺-F cation [P. Kirsch, Modern Fluoroorganic Chemistry. Synthesis, Reactivity, Applications; Chapter 2.1.6., page 73ff, WILEY-VCH, 2004], whereas the influence of the anion (BF₄⁻, PF₆⁻ or CF₃SO₃⁻) is rather low. As explained below, however, it has been shown in the present invention that a suitable anion is entirely capable of crucially influencing the fluorination force.

However, the reagents F-TEDA-BF₄ and NFPy-BF₄ also have disadvantages. Thus, for example, the solubility of F-TEDA-BF₄ in acetonitrile is limited and in common organic solvents, such as lower alcohols, acetone or dichloromethane, is very low [R. P. Singh, J. M. Shreeve, 2004, Acc. Chem. Res. 37: 31-44; P. T. Nyffeler, S. G. Duron, M. D. Burkart R. P. Singh, J. M. Shreeve, St. P. Vincent, Ch. -H. Wong, 2005, Angew. Chem. 117: 196-217], which restricts the practical use of this fluorinating reagent. In addition, large amounts of F-TEDA-BF₄ should be stored in a cool, dry place and should not be heated to temperatures above 80° C. [R. P. Singh, J. M. Shreeve, 2004, Acc. Chem. Res. 37: 31-44]. The guaranteed storage stability is usually not more than one year.

The object of the present invention was thus the provision of novel fluorinating reagents having improved properties, for example with respect to their solubility or their stability.

Surprisingly, it has now been found that NF fluorinating reagents having improved properties can be obtained if the anion is replaced by an FAP anion ((perfluoroalkyl)fluorophosphate anion).

The present invention therefore relates firstly to the use of a compound of the formula (I)

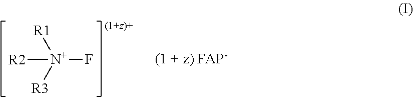

as fluorinating reagent,
where R1, R2 and R3 each, independently of one another, stand for
straight-chain or branched alkyl having 1-20 C atoms,
saturated, partially or fully unsaturated cycloalkyl having 3-7 C atoms,

which may be substituted by alkyl groups having 1-6 C atoms, where one or two radicals selected from R1, R2 and R3 may be fully substituted and/or one or more radicals selected from R1, R2 and R3 may be partially substituted by halogens or partially by —OR¹, —NR¹*₂, —CN, —C(O)NR¹₂or —SO₂NR¹₂,
where one or two non-adjacent carbon atoms which are not in the α-position of the radicals R1, R2 and/or R3 may be replaced by atoms and/or atom groups selected from the group —O—, —SO₂—, —N⁺R¹₂—, —C(O)NR¹— or —SO₂NR¹₂—,
and where the radicals R1, R2 and/or R3 may be linked to one another and may form a heterocyclic ring system with the nitrogen atom,
in which R1 stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl,
and where FAP⁻ stands for an anion of the formula (II)
   [P(CₙF₂ₙ₊₁)_{y}F_{6-y}]⁻(II)
where n=1 to 8,
   y=1 to 4,
and z stands for 0, 1 or 2.

A straight-chain or branched alkyl having 1-20 C atoms is taken to mean, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl or dodecyl. As described above, these radicals may be partially substituted by halogens, in particular by —F and/or —Cl, or partially by —OR¹, —NR¹*₂, —CN, —C(O)NR¹₂, —SO₂NR¹₂.

Saturated or partially or fully unsaturated cycloalkyl groups having 3-7 C atoms are, for example, cyclopropyl, cyclobutyl, cyclopentyl, cyclohexyl, cycloheptyl, cyclopentenyl, cyclohexenyl, phenyl, cycloheptenyl, each of which may be substituted by C₁- to C₆-alkyl groups, where the cycloalkyl group or the cycloalkyl group substituted by C₁ - to C₆-alkyl groups may in turn also be substituted by halogen atoms, such as F, Cl, Br or I, in particular F or Cl, or by —OR¹, —NR¹*₂, —CN, —C(O)NR¹₂, —SO₂NR¹₂.

In the substituents of R1, R2 and R3, R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl.

Without restricting generality, examples of substituents of R1, R2 and R3are therefore:
—OCH₃, —OCH(CH₃₎₂, —CH₂OCH₃, —CH₂-CH₂-O-CH₃, —C₂H₄OCH(CH₃)₂, —SO₂CH₃, —SO₂C₆H₅, —SO₂C₃H₇, —SO₂CH(CH₃)₂, —SO₂CH₂CF₃, —CH₂SO₂CH₃, —O-C₄H₈-O-C₄H₉, —CF₃, —C₂F₅, —C₃F₇, —C₄F₉, —C(CF₃)₃, —CF₂SO₂CF₃, —C₂F₄N(C₂F₅)C₂F₅, —CHF₂, —CH₂CF₃, —C₂F₂H₃, —C₃FH₆, —CH₂C₃F₇, —C(CFH₂)₃ or —CH₂C₆H₅.

In R¹ and R¹*, substituted phenyl denotes phenyl which is substituted by C₁- to C₆-alkyl, C₁- to C₆-alkenyl, CN, NR¹₂, F, Cl, Br, I, C₁-C₆-alkoxy, SCF₃, SO₂CF₃ or SO₂NR*₂, where R* denotes a non-, partially or perfluorinated C₁- to C₆-alkyl or C₃- to C₇-cycloalkyl as defined for R¹, for example, o-, m- or p-methylphenyl, o-, m- or p-ethylphenyl, o-, m- or p-propylphenyl, o-, m- or p-isopropylphenyl, o-, m- or p-tert-butylphenyl, o-, m- or p-methoxyphenyl, o-, m- or p-ethoxyphenyl, o-, m-, p-(trifluoromethyl)phenyl, o-, m-, p-(trifluoromethoxy)phenyl, o-, m-, p-(trifluoromethylsulfonyl)phenyl, o-, m- or p-fluorophenyl, o-, m- or p-chlorophenyl, o-, m- or p-bromophenyl, o-, m- or p-iodophenyl, further preferably 2,3-, 2,4-, 2,5-, 2,6-, 3,4- or 3,5-dimethylphenyl, 2,3-, 2,4-, 2,5-, 2,6-, 3,4- or 3,5-dihydroxyphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

R1, R2 and/or R3 preferably form a heterocyclic ring system with the nitrogen atom. Saturated or unsaturated ring systems of this type include mono- and bicyclic and aromatic ring systems in which two of the three radicals R1, R2 or R3 represent a common radical, which is connected to the nitrogen atom via a double bond. As an example thereof, mention may be made of structures containing a pyridine skeleton.

A saturated or unsaturated mono- or bicyclic heterocyclic radical may contain 5 to 13 ring members, where 1, 2 or 3 N and 1 or 2 S or O atoms may be present and the heterocyclic radical may be mono- or polysubstituted by C₁- to C₆-alkyl, CN, NR¹*₂, F, Cl, Br, I, C₁-C₆-alkoxy, SO₂CF₃ or SO₂NR¹₂, where R¹ and R¹* have a meaning indicated above.

The heterocyclic radical is preferably substituted or unsubstituted 1-, 2- or 3-pyrrolyl, 1-, 2-, 4- or 5-imidazolyl, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1 -, -4- or -5-yl, 1 - or 5-tetrazolyl, 1,2,3-oxadiazol-4- or -5-yl 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1 -, 2-, 3-, 4-, 5-, 6- or 7-1H-indolyl, 1 -, 2-, 4- or 5-benzimidazolyl, 1 -, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 1-, 2-, 3-, 4-, 5-, 6-, 7- or 8-quinolinyl, 1-, 3-, 4-, 5-, 6-, 7- or 8-isoquinolinyl, 1-, 2-, 3-, 4- or 9-carbazolyl, 1-, 2-, 3-, 4-, 5-, 6-, 7-, 8- or 9-acridinyl, 3-, 4-, 5-, 6-, 7- or 8-cinnolinyl, 2-, 4-, 5-, 6-, 7- or 8-quinazolinyl or 1-, 2- or 3-pyrrolidinyl.

The compound of the formula (I) particularly preferably stands for a compound of the formula (III)

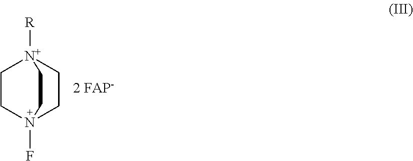

where R stands for
straight-chain or branched alkyl having 1-20 C atoms,
saturated, partially or fully unsaturated cycloalkyl having 3-7 C atoms,

which may be substituted by alkyl groups having 1-6 C atoms, where R may be fully or partially substituted by halogens or partially by —OR¹, —NR¹*₂, —CN, —C(O)NR¹₂ or —SO₂NR¹₂,
and where one or two non-adjacent carbon atoms of R which are not in the α-position may be replaced by atoms and/or atom groups selected from the group —O—, —SO₂—, —N⁺R¹₂—, —C(O)NR₁- or —SO₂NR¹-, in which R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl.

R in formula (III) preferably stands for a straight-chain or branched alkyl having 1-4 C atoms, where R may be partially substituted by halogens, such as F, Cl, Br or I, in particular Cl.

Examples of a straight-chain or branched alkyl having 1-4 C atoms are methyl, ethyl, isopropyl, propyl, butyl, sec-butyl or tert-butyl. These radicals may be partially substituted, as described above, by halogens, in particular by —Cl. Examples thereof are —CHCl₂, —CH₂Cl, —CH₂CCl₃, —C₂Cl₂H₃, —C₃ClH₆, —CH₂C₃Cl₇ or —C(CClH₂)₃.

In a very particularly preferred embodiment, the compound of the formula (I) stands for a compound of the formula (IV)

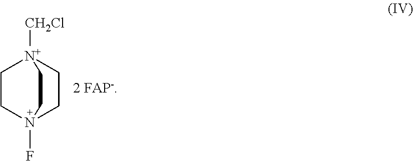

In a further very particularly preferred embodiment, the compound of the formula (I) stands for a compound of the formula (V)

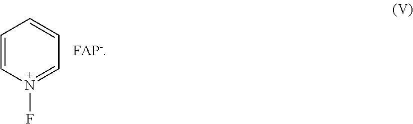

In the present invention, FAP⁻ preferably stands for an anion of the formula (II)
[P(CₙF₂ₙ₊₁)_{y}F_{6-y}]⁻ (II)
where n=1 to 4,
   y=1 to 3.

FAP⁻ particularly preferably stands for [(CF₃)₃PF₃]⁻, [(C₂F₅)₃PF₃]⁻, [(C₃F₇)₃PF₃]⁻, [(C₄F₉)₃PF₃]⁻, [(CF₃)₂PF₄]⁻, [(C₂F₅)₂PF₄]⁻, [(C₃F₇)₂PF₄]⁻ or [(C₄F₉)₂PF₄]⁻, especially preferably for [(C₂F₅)₃PF₃]⁻.

In a particularly advantageous embodiment of the use according to the invention, the compound of the formula (I) stands for a compound of the formula (IVa)

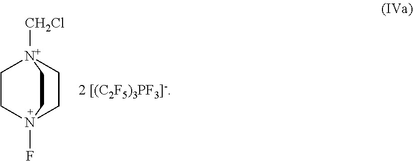

In a further particularly advantageous embodiment, the compound of the formula (I) stands for a compound of the formula (Va)

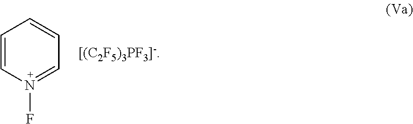

The use according to the invention of a compound of the formula (I) as fluorinating reagent has various advantages over the use of corresponding fluorinating reagents having a BF₄⁻ anion: the compounds of the formula (I) have improved stability and can therefore be stored over a longer period. In addition, the solubility of the compounds in certain organic solvents, such as acetonitrile, dichloromethane, dialkyl ethers, ethylene glycol dimethyl ether (monoglyme), bis(2-methoxyethyl) ether (diglyme) or N,N-dimethylacetamide, is increased. The area of use of these fluorinating reagents is thus broader. A further advantage on use of fluorinating reagents of the formula (I) arises through their significantly higher fluorination force. This results in a greatly increased reaction rate, even at room temperature.

The present invention furthermore relates to a process for the fluorination of a nucleophilic organic compound, characterised in that the nucleophilic organic compound is reacted with a compound of the formula (I)

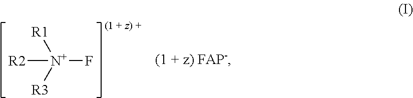

where R1, R2 and R3 each, independently of one another, stand for
straight-chain or branched alkyl having 1-20 C atoms,
saturated, partially or fully unsaturated cycloalkyl having 3-7 C atoms,

which may be substituted by alkyl groups having 1-6 C atoms, where one or two radicals selected from R1, R2 and R3 may be fully substituted and/or one or more radicals selected from R1, R2 and R3 may be partially substituted by halogens or partially by —OR¹, —NR¹*₂, —CN, —C(O)NR¹₂or —SO₂NR¹₂,
where one or two non-adjacent carbon atoms which are not in the α-position of the radicals R1, R2 and/or R3 may be replaced by atoms and/or atom groups selected from the group —O—, —SO₂—, —N⁺R¹₂—, —C(O)NR¹- or —SO₂NR¹-,
and where the radicals R1, R2 and/or R3 may be linked to one another and may form a heterocyclic ring system with the nitrogen atom,
in which R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl,
FAP⁻ stands for an anion of the formula (II)
   [P(CₙF₂ₙ₊₁)_{y}F_{6-y}]⁻ (II)
      where n=1 to 8
   y=1 to 4,
      and z stands for 0, 1 or 2.

In the preferred embodiments of the process according to the invention, R1, R2 and R3, as well as FAP⁻ are defined as described above.

In a preferred embodiment of the process according to the invention, the reaction is carried out in an organic solvent. Any standard organic solvent can be employed for this purpose. The choice of a suitable solvent is dependent on the organic compound to be reacted and can readily be made by a person skilled in the art. Examples of suitable organic solvents are acetonitrile, N,N-dimethylacetamide, ethylene glycol dimethyl ether (monoglyme) or dichloromethane.

Acetonitrile is particularly preferably employed.

In a further preferred embodiment of the process according to the invention, the reaction is carried out in a solvent-free medium.

In the process according to the invention, the reaction is preferably carried out at a temperature between −20° C. to 120° C., a temperature of 10° C. to 40° C. is particularly preferred. The reaction is very particularly advantageously carried out at room temperature, since fluorinating reagents of the formula (I) are particularly stable and active at room temperature.

Nucleophilic organic compounds which are suitable for the process according to the invention are compounds containing electron-rich centres, which are able to react with electrophiles. These include, for example, compounds with unsaturated carbon compounds in general, activated olefins (aryl-substituted alkenes, alkyl and silylenol ethers, enol esters and enamines), activated aromatic compounds, thioglycosides, thioethers, heterocyclic compounds, stabilised carbanions, certain organometallic compounds and aliphatic sulfides, disulfides and selenides.

Aromatic compounds having one or more electron-donating substituents are particularly preferably employed. Electron-donating substituents are taken to mean functional groups which are able to exert a+l effect, i.e. a positive inductive effect, via a σ bond (for example methyl groups), or a +M effect, i.e. a positive mesomeric effect, via p-π conjugation (for example alkoxy or dialkylamino groups).

Examples thereof are anisole, phenetol, N,N-dimethylaniline.

A preferred embodiment of the process according to the invention is characterised in that the nucleophilic organic compound is 1,3-diphenyl-1,3-propanedione.

The present invention also relates to compounds of the formula (III)

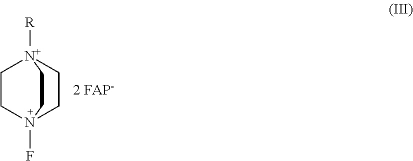

where R stands for
straight-chain or branched alkyl having 1-20 C atoms,
saturated, partially or fully unsaturated cycloalkyl having 3-7 C atoms,

which may be substituted by alkyl groups having 1-6 C atoms, where R may be fully or partially substituted by halogens or partially by —OR¹, —NR¹*₂, —CN, —C(O)NR¹₂ or —SO₂NR¹₂,

and where one or two non-adjacent carbon atoms of R which are not in the α-position may be replaced by atoms and/or atom groups selected from the group —O—, —SO₂—, —N⁺R¹₂—, —C(O)NR¹- or —SO₂NR¹-, in which R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl,
and where FAP⁻ stands for an anion of the general formula (II)
[P(CₙF₂ₙ₊₁)_{y}F_{6-y}]⁻ (II)
   where n=1 to 8,
y=1 to 4.

In formula (III) of the compounds according to the invention, R preferably stands for a straight-chain or branched alkyl having 1-4 C atoms, where R may be fully or partially substituted by halogens.

The compounds of the formula (III) are particularly preferably compounds of the formula (IV)

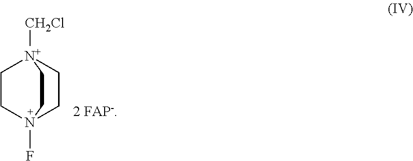

The present invention likewise relates to compounds of the formula (V)

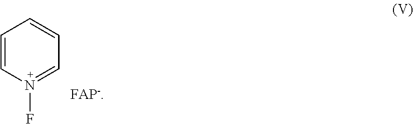

In the formulae (IV) and (V), FAP⁻ stands for an anion of the general formula (II)
[P(CₙF₂ₙ₊₁)_{y}F_{6-y}]⁻ (II)
   where n=1 to 8,
y=1 to 4.

FAP⁻ in the compounds of the formulae (III), (IV) and (V) preferably stands for an anion of the formula (II)
[P(CₙF₂ₙ₊₁)_{y}F_{6-y}]⁻ (II)
   where n=1 to 4,
y=1 to 3.

FAP⁻ in the compounds of the formulae (III), (IV) and (V) particularly preferably stands for [(CF₃)₃PF₃]⁻, [(C₂F₅)₃PF₃]⁻, [(C₃F₇)₃PF₃]⁻, [(C₄F₉)₃PF₃]⁻, [(CF₃)₂PF₄]⁻, [(C₂F₅)₂PF₄]⁻, [(C₃F₇)₂PF₄]⁻ or [(C₄F₉)₂PF₄]⁻, especially preferably for [(C₂F₅)₃PF₃]⁻.

The compounds according to the invention are particularly preferably selected from the group comprising the compounds (IVa) and (Va)

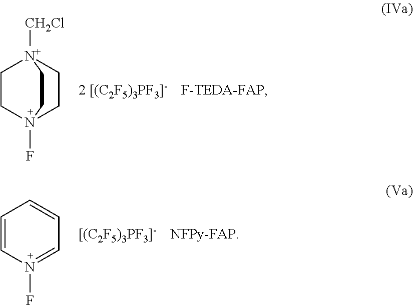

F-TEDA-FAP and NFPy-FAP can be prepared analogously to F-TEDA-BF₄ or NFPy-BF₄. This is depicted by way of example below:

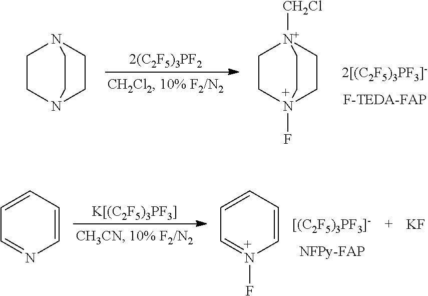

F-TEDA-FAP and NFPy-FAP can also be prepared starting from F-TEDA-BF₄ and NFPy-BF₄ respectively with the aid of an anion exchange reaction by K[(C₂F₅)₃PF₃] (KFAP, available from Merck KGaA).

F-TEDA-FAP and NFPy-FAP have not only improved stability and higher solubility in organic solvents compared with F-TEDA-BF₄ and NFPy-BF₄, but also have a significantly higher fluorination force. This is illustrated with reference to Examples 3 and 4.

The following working examples are intended to explain the invention without limiting it. The invention can be carried out correspondingly throughout the range claimed. Possible variants can also be derived starting from the examples. Thus, the features and conditions of the reactions described in the examples can also be applied to other reactions which are not described in detail, but fall within the scope of protection of the claims.

### EXAMPLES

The ¹H and ¹⁹F NMR spectra are recorded on a Brucker Avance 300 spectrometer (resonance frequency for ¹H: 300.13 MHz, for ¹⁹F: 282.40 MHz) in acetonitrile-D3. CCl₃F and TMS serve as reference substance for the ¹⁹F and ¹H NMR spectra respectively.

TABLE 1
Comparison of the solubility of F-TEDA-BF₄ and F-TEDA-FAP
(compound (IVa)); NFPy-BF₄ and NFPy-FAP (compound (Va)).

| Compound | Solvent | Solubility |
|---|---|---|
| F-TEDA-BF₄ | CH₃CN | 1.2% |
| | CH₂Cl₂ | insoluble |
| | benzene | insoluble |
| F-TEDA-FAP (IVa) | CH₃CN | 30% |
| | CH₂Cl₂ | insoluble |
| | benzene | sparingly soluble |
| NFPy-BF₄ | CH₃CN | 4.3% |
| | CH₂Cl₂ | insoluble |
| | benzene | insoluble |
| NFPy-FAP (Va) | CH₃CN | 34% |
| | CH₂Cl₂ | 0.4% |
| | benzene | insoluble |

### Example 1

### Preparation of 1-chloromethyl-4-fluorodiazoniabicyclo[2.2.2]octane bis[tris- (pentafluoroethyl)trifluorophosphate], F-TEDA-FAP (compound IVa)

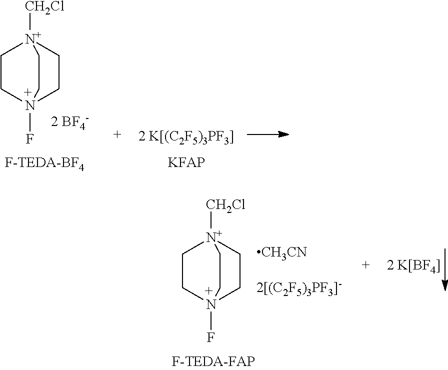

A solution of 0.56 g (1.16 mmol) of KFAP in 5 cm³ of CH₃CN is added to a solution of 0.21 g (0.58 mmol) of F-TEDA-BF4 in 50 cm³ of CH₃CN at room temperature. The reaction mixture is stirred at room temperature for three hours. The white precipitate is subsequently filtered off. Evaporation of the solvent gives 0.57 g of a white solid. The yield of F-TEDA-FAP is 88% (melting point 109° C.). The compound is investigated by NMR spectroscopy and elemental analysis.

¹H NMR (in CD₃CN, reference substance: TMS), δ[ppm]:

2.1 s (CH₃), 4.2 t (6H; 3,5,8-CH₂), 4.7 t (6H; 2,6,7-CH₂), 5.3 s (CH₂Cl).

¹⁹F NMR (in CD₃CN, reference substance: CCl₃F), δ[ppm]:

49.4 m (FN⁺), −43.2 d, m (PF), −79.3 m (CF₃), −81.0 m (2CF₃), −86.5 d, m (PF₂), −114.7 d, m (CF₂), −115.3 d, m (2CF₂).

### Elemental Analysis

Found: C 22.65%, H 1.54%, N 3.82%

Calculated for F-TEDA-FAP•CH₃CN: C 22.69%, H 1.54%, N 3.78%

The structure of F-TEDA-FAP•CH₃CN is confirmed by X-ray structural analysis.

### Example 2

### Preparation of N-fluoropyridinium tris(pentafluoroethyl)trifluorophosphate NFPy-FAP (compound (Va))

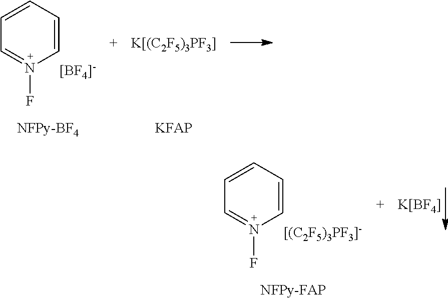

A solution of 0.52 g (1.08 mmol) of KFAP in 10 cm³ of CH₃CN is added to a solution of 0.20 g (1.08 mmol) of NFPy-BF₄ in 5 cm³ of CH₃CN at room temperature. The reaction mixture is stirred at room temperature for 15 min and subsequently cooled to −20° C. The white precipitate is filtered off. Evaporation of the solvent gives 0.54 g of a white solid. The yield of NFPy-FAP is 91% (melting point: 66° C.). The compound was investigated by NMR spectroscopy and elemental analysis:

¹H NMR (in CD₃CN, reference substance: TMS), δ[ppm]:

8.3 m (2H; 3,5-CH), 8.7 m (1H; 4-CH), 9.2 m (2H; 2,6-CH).

¹⁹F NMR (in CD₃CN, reference substance: CCl₃F), δ[ppm]:

47.9 m (FN⁺), −43.3 d, m (PF), −79.3 m (CF₃), −81.0 m (2CF₃), −87.0 d, m (PF₂), −114.8 d, m (CF₂), −115.4 d, m, (2CF₂).

### Elemental Analysis

Found: C 24.30%, H 0.83%, N 2.72%

Calculated for NFPy-FAP: C 24.33%, H 0.93%, N 2.58%

### Example 3

### Solvent-free fluorination of 1,3-diphenyl-1,3-propanedione with the aid of F-TEDA-FAP (compound (IVa)) compared with F-TEDA-BF₄:

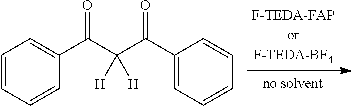 [SMILES: O=C(CC(=O)c1ccccc1)c1ccccc1]

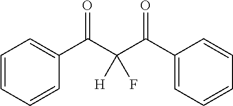 [SMILES: O=C(c1ccccc1)C(F)C(=O)c1ccccc1]

| | Reaction | Product, | Product, | |
|---|---|---|---|---|
| | time | ¹⁹F-NMR | ¹H-NMR | Yield |
| F-TEDA-BF₄ | 3 hours | −185.2 ppm d, | 6.58 ppm d, | 77% |
| | | J_{H,F} = 49.4 Hz | J_{H,F} = 49.1 Hz | |
| F-TEDA-FAP | 10 min | −186.9 ppm d, | 6.58 ppm d | 84% |
| | | J_{H,F} = 49.1 Hz | J_{H,F} = 49.1Hz | |

### Example 4

### Solvent-free fluorination of 1,3-diphenyl-1,3-propanedione using NFPy-FAP (compound (Va)) compared with NFPy-BF₄

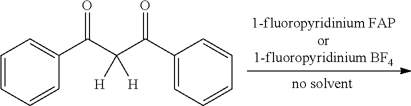 [SMILES: O=C(CC(=O)c1ccccc1)c1ccccc1]

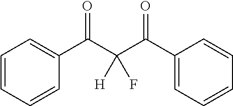 [SMILES: O=C(c1ccccc1)C(F)C(=O)c1ccccc1]

| | Reaction | Product, | Product, | |
|---|---|---|---|---|
| | time | ¹⁹F-NMR | ¹H-NMR | Yield |
| NFPy-BF₄ | 5 hours | Product is not | 6.87 ppm s, | Product is not |
| | | detected | (starting mater- | detected |
| | | | ial) | |
| NFPy-FAP | 10 min | −185.2 ppm d, | 6.58 ppm d, | 34% |
| | | J_{H,F} = 49.1 Hz | J_{H,F} = 49.1 Hz | |

1. A process for the fluorination of a compound which comprises using a compound of the formula (I) 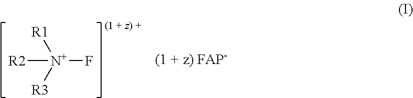
as a fluorinating reagent,
where R1, R2 and R3 each, independently of one another, stand for
straight-chain or branched alkyl having 1-20 C atoms,
saturated, partially or fully unsaturated cycloalkyl having 3-7 C atoms,
which may be substituted by alkyl groups having 1-6 C atoms, where one or two radicals selected from R1, R2 and R3 may be fully substituted and/or one or more radicals selected from R1, R2 and R3 may be partially substituted by halogens or partially by —OR¹, —NR¹*₂, —CN, —C(O)NR¹₂ or —SO₂NR¹₂, where one or two non-adjacent carbon atoms which are not in the α-position of the radicals R1, R2 and/or R3 may be replaced by atoms and/or atom groups selected from the group —O—, —SO₂—, —N⁺R¹₂—, —C(O)NR¹- or —SO₂NR¹—,
and where the radicals R1, R2 and/or R3 may be linked to one another and may form a heterocyclic ring system with the nitrogen atom,
in which R1 stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl,
and where FAP⁻ stands for an anion of the formula (II)
[P(CₙF₂ₙ₊₁)_{y}F_{6-y}]⁻ (II)
where n=1 to 8,
y=1 to 4,
and z stands for 0, 1 or 2.

2. A process according to claim 1, characterised in that R1, R2 and/or R3 form a heterocyclic ring system with the nitrogen atom.

3. A process according to claim 1, characterised in that the compound of the formula (I) stands for a compound of the formula (III) 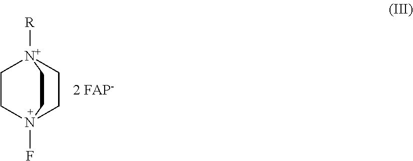
where R stands for
straight-chain or branched alkyl having 1-20 C atoms,
saturated, partially or fully unsaturated cycloalkyl having 3-7 C
atoms, which may be substituted by alkyl groups having 1-6 C atoms,
where R may be fully or partially substituted by halogens or partially by —OR¹, —NR¹*₂, —CN, —C(O)NR¹₂ or —SO₂NR¹-,
and where one or two non-adjacent carbon atoms of R which are not in the α-position may be replaced by atoms and/or atom groups selected from the group —O—, —SO₂-, —N⁺R¹₂—, —C(O)NR¹- or —SO₂NR¹-,
in which R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl.

4. A process according to claim 1, characterised in that the compound of the formula (I) stands for a compound of the formula (IV)

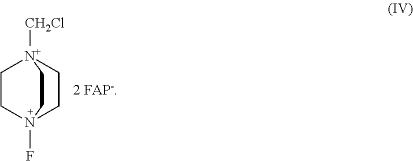

5. A process according to claim 1, characterised in that the compound of the formula (I) stands for a compound of the formula (V)

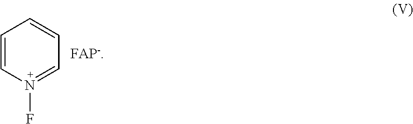

6. A process according to claim 1, characterised in that FAP⁻ stands for an anion of the formula (II)
[P(CₙF₂ₙ₊₁)_{y}F_{6-y}]⁻ (II)
where n=1 to 4,
y=1 to 3.

7. A process according to claim 1, characterised in that FAP⁻ stands for [(CF₃)₃PF₃]⁻, [(C₂F₅)₃PF₃]⁻, [(C₃F₇)₃PF₃]⁻, [(C₄F₉)₃PF₃]⁻, [(CF₃)₂PF₄]⁻, [(C₂F₅)₂PF₄]⁻, [(C₃F₇)₂PF₄]⁻ or [(C₄F₉)₂PF₄]⁻.

8. A process for the fluorination of a nucleophilic organic compound, wherein said nucleophilic organic compound is reacted with a compound of the formula (I) 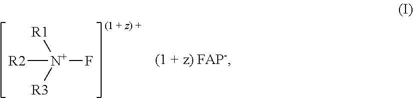
where R1, R2 and R3 each, independently of one another, stand for
straight-chain or branched alkyl having 1-20 C atoms,
saturated, partially or fully unsaturated cycloalkyl having 3-7 C
atoms, which may be substituted by alkyl groups having 1-6 C atoms,
where one or two radicals selected from R1, R2 and R3 may be fully substituted and/or one or more radicals selected from R1, R2 and R3 may be partially substituted by halogens or partially by —OR¹, —NR¹*₂, —CN, —C(O)NR¹₂ or —SO₂NR¹₂,
where one or two non-adjacent carbon atoms which are not in the α-position of the radicals R1, R2 and/or R3 may be replaced by atoms and/or atom groups selected from the group —O—, —SO₂—, —N⁺R¹₂—, —C(O)NR¹- or —SO₂NR¹-,
and where the radicals R1, R2 and/or R3 may be linked to one another and may form a heterocyclic ring system with the nitrogen atom,
in which R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl,
FAP⁻ stands for an anion of the formula (II)
[P(CₙF₂ₙ₊₁)_{y}F_{6-y}]⁻ (II)
where n=1 to 8
y=1 to 4,
and z stands for 0, 1 or 2.

9. A process according to claim 8, characterised in that the reaction is carried out in an organic solvent.

10. A process according to claim 8, characterised in that the reaction is carried out in a solvent-free medium.

11. A process according claim 8, characterised in that the reaction is carried out at a temperature of −20° C. to 120° C.

12. A process according to claim 8, characterised in that the nucleophilic organic compound is an aromatic compounds having one or more electron-donating substituents.

13. A compound of the formula (III) 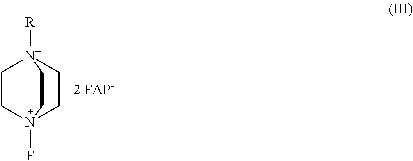
where R stands for
straight-chain or branched alkyl having 1-20 C atoms,
saturated, partially or fully unsaturated cycloalkyl having 3-7 C atoms, which may be substituted by alkyl groups having 1-6 C atoms,
in which R may be fully or partially substituted by halogens or partially by —OR¹, —NR¹*₂, —CN, —C(O)NR¹₂ or —SO₂NR¹₂,
and where one or two non-adjacent carbon atoms of R which are not in the α-position may be replaced by atoms and/or atom groups selected from the group —O—, —SO₂—, —N⁺R¹₂—, —C(O)NR¹- or —SO₂NR¹-,
where R¹ stands for H, non-, partially or perfluorinated C₁- to C₆-alkyl, C₃- to C₇-cycloalkyl, unsubstituted or substituted phenyl, and R¹* stands for non-, partially or perfluorinated C₁- to C₆-alkyl, C₃- to C₇-cycloalkyl, unsubstituted or substituted phenyl,
and where FAP⁻ stands for an anion of the general formula (II)
[P(CₙF₂ₙ₊₁)_{y}F_{6-y}]⁻ (II)
where n=1 to 8,
y=1 to 4.

14. A compound according to claim 13, characterised in that the compound of the formula (III) stands for a compound of the formula (IV) 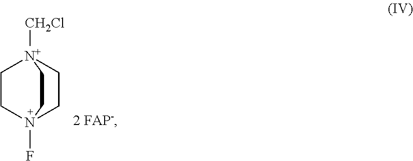
where FAP⁻ stands for an anion of the general formula (II)
[P(CₙF₂ₙ₊₁)_{y}F_{6-y}]⁻ (II)
where n=1 to 8,
y=1 to 4.

15. A compound of the formula (V) 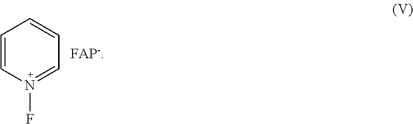
where FAP⁻ stands for an anion of the general formula (II)
[P(CₙF₂ₙ₊₁)_{y}F_{6-y}]⁻ (II)
where n=1 to 8,
y=1 to 4.
